(19) United States
(12) Patent Application Publication
(10) Pub. No.: US 2001/0023298 A1
(43) Pub. Date: Sep. 20, 2001

(54) Process for preparing fatty acid N-alkylpolyhydroxyamides

(75) Inventor: Frank Weinelt, Burgkirchen (DE)

Correspondence Address: CLARIANT CORPORATION, 4331 CHESAPEAKE DR, ATTN: INDUSTRIAL PROPERTY DEPT, CHARLOTTE, NC, 28216, US

(73) Assignee: Clariant GmbH

(21) Appl. No.: 09/742,823

(22) Filed: Dec. 21, 2000

(30) Foreign Application Priority Data
Dec. 24, 1999 (DE) 19962999.4

(51) Int. Cl. C07C 233/02

(52) U.S. Cl. 554/61

(57) ABSTRACT

A process for preparing fatty acid N-alkylpolyhydroxyamides of the formula

R¹CONR²Z

in which R¹ is a linear or branched, saturated or unsaturated alkyl group having 7 to 21 carbon atoms, Z is a polyhydroxyhydrocarbon group having at least three hydroxyl groups, which can also be alkoxylated, and R² is H, C₁-C₈-alkyl, a group of the formula —(CH₂)ₓNR³R⁴ or R⁵O(CH₂)ₙ—, R³ and R⁴ are C₁-C₄-alkyl or C₂-C₄-hydroxyalkyl, R⁵ is C₁-C₄-alkyl, n is a number from 2 to 4 and x is a number from 2 to 10, by reacting an N-alkyl-polyhydroxyamine of the formula

H—NR²Z

with a fatty acid, fatty acid chloride or fatty acid ester in the presence of a basic catalyst, which comprises carrying out the reaction in the presence of a fatty acid amidoalkoxylate of the formula

R—CONR⁶R⁷

where R is alkyl or alkenyl having 7 to 21 carbon atoms, R⁶ is hydrogen or a group —(AO)ₓH, R⁷ is a group —(AO)ₓH, A is a group of the formula —C₂H₄—, —C₃H₆— or —C₄H₈— and x is a number from 1 to 20.

### BACKGROUND OF THE INVENTION

(1) Field of the Invention

The invention relates to an improved process for preparing fatty acid N-alkylpolyhydroxyamides of the formula

R¹CONR²Z (I)

in which R¹ is a linear or branched, saturated or unsaturated alkyl group having 7 to 21 carbon atoms, Z is a polyhydroxyhydrocarbon group having at least three hydroxyl groups, which can also be alkoxylated, and R² is H, C₁-C₈-alkyl, a group of the formula —(CH₂)ₓNR³R⁴ or R⁵O(CH₂)ₙ—, R³ and R⁴ are C₁-C₄-alkyl or C₂-C₄-hydroxyalkyl, R⁵ is C₁-C₄-alkyl, n is a number from 2 to 4 and x is a number from 2 to 10.

Fatty acid N-alkylpolyhydroxyamides are nonionic surfactants based on renewable raw materials which, owing to their excellent use properties and their particular ecotoxicological compatibility are gaining increasing importance for the preparation of surfactants, but also for cosmetic products, and in crop protection compositions etc.

The compounds of the formula I are, as described in WO 92/06160, or in the literature stated therein, prepared by reductive amination of a reducing sugar with ammonia, an alkylamine or an alkanolamine, to give an N-alkylpolyhydroxyamine of the formula II

H—NR²Z (II)

where Z and R² are as defined above, followed by acylation with a fatty acid, fatty acid chloride or fatty acid ester. For the acylation of the amines of the formula II, the amines are melted at temperatures of preferably from 125 to 130° C. and reacted with the acylating agent. In addition to the high energy input, the formation of undesirable byproducts, in particular cyclic sugar derivatives, is disadvantageous here.

### SUMMARY OF THE INVENTION

Accordingly, it was the object to develop an improved process for preparing this class of surfactant.

Surprisingly, it has been found that the synthesis of fatty acid N-alkylpolyhydroxyamides of the formula I by acylation of the amines of the formula II in the presence of a basic catalyst can be carried out in shorter reaction times if fatty acid amidoalkoxylates of the formula III are added to the N-alkylpolyhydroxyamine of the formula II.

R—CON(R⁶)(R⁷) (III)

in which R is alkyl or alkenyl having 7 to 21 carbon atoms, R⁶ is hydrogen or a group —(AO)ₓH, R⁷ is a group —(AO)ₓH, A is a group of the formula —C₂H₄—, —C₃H₆— or —C₄H₈— and x is a number from 1 to 20.

The addition of fatty acid amidoalkoxylate leads to a reduction of the reaction times. This behavior is not observed when fatty alcohol ethoxylates are used in place of fatty acid amidoalkoxylates.

### DESCRIPTION OF THE PREFERRED EMBODIMENTS

The process according to the invention for preparing fatty acid N-alkylpolyhydroxyalkylamides of the formula I represents a significant economical advantage and additionally results in advantages on use, such as improved solubility and more favorable viscosity behavior of the product mixture of amide of the formula I and fatty acid amidoalkoxylate of the formula III.

Fatty acid N-alkylpolyhydroxyalkylamides which are preferably preferred are derived from reducing sugars having 5 or 6 carbon atoms, in particular from glucose.

Accordingly, the fatty acid N-alkylpolyhydroxyalkylamides which are preferably prepared are fatty acid N-alkylglucamides represented by the formula IV

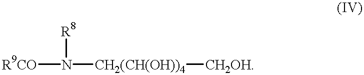

The fatty acid N-alkylpolyhydroxyalkylamides which are preferably prepared are glucamides of the formula IV in which R⁸ is a C₁-C₄-alkyl group, in particular methyl, and R⁹CO is the radical of a saturated or unsaturated fatty acid having 8 to 22 carbon atoms. In particular, R⁹CO is the acyl radical of caproic acid, caprylic acid, capric acid, lauric acid, myristic acid, palmitic acid, palmoleic acid, stearic acid, isostearic acid, oleic acid, elaidic acid, petroselic acid, linoleic acid, linolenic acid, arachidic acid, gadoleic acid, behenic acid or erucic acid, tallow fatty acid or technical-grade mixtures thereof, particularly preferably C₁₂-C₁₈-, in particular C₁₂-C₁₄- and C₁₆-C₁₈-fatty acids.

Furthermore, the polyhydroxyalkylamides prepared by the process according to the invention can also be derived from maltose and palatinose.

The fatty acid amidoalkoxylates of the formula III employed in the process according to the invention for preparing fatty acid N-alkylpolyhydroxyalkylamides are obtained by reacting fatty acid methyl esters and mono- or dialkanolamine, followed by alkoxylation.

The preparation according to the invention of the fatty acid N-alkylpolyhydroxy-amides is preferably carried out as follows:

according to customary processes, N-alkylpolyhydroxyamine is dewatered in a stripper in the temperature range of from 95 to 135° C. at atmospheric pressure and then at reduced pressure in the range from 1000 mbar to 20 mbar, preferably from 30 mbar, until the residual water content is from 0.01 to 0.5% by weight, preferably 0.2% by weight.

The amine, melted at a temperature of from 127 to 130° C., is admixed with stirring with fatty acid amidoalkoxylate, heated to from 80 to 100° C., in amounts of from 10 to 70% by weight, preferably from 15 to 40% by weight, particularly preferably from 20 to 30% by weight, based on the total reaction mixture, and the acylating agent fatty acid or fatty acid chloride, in particular fatty acid alkyl ester, in amounts of from 100 to 120 mol %, preferably from 105 to 115 mol %, particularly preferably from 105 to 110 mol %, based on the amount of amine, and the temperature of the mixture is kept above the solidification point. The melting point of the mixture is 5 to 7° C. lower than the melting point of the amine. Over a period of from 5 to 10 minutes from 2 to 15 mol %, preferably from 5 to 10 mol %, based on the amount of amine, of a basic catalyst, for example a metal alkoxide, in particular sodium methoxide, or an alkali metal salt, in particular the sodium salt of glycerol or propylene glycol, preferably dissolved in an organic solvent, preferably methanol, or an alkali metal hydroxide, in particular NaOH, are then added dropwise.

Within 5 to 10 minutes, the reaction sets in with formation of alcohol (or with formation of H₂0 or HCl) which is distilled off at from 120° C. to 100° C. under atmospheric pressure, then at from 100° C. to 80° C., preferably at from 90° C. to 85° C., under reduced pressure at from 400 mbar to 20 mbar, preferably from 100 mbar to 20 mbar. Simultaneously with the formation of alcohol, it can be observed that the reaction mixture becomes clear.

The fatty acid amidoalkoxylates added in the process according to the invention for preparing fatty acid N-alkylpolyhydroxyamides cause a reduction of the start-up phase to the reaction of amine with fatty acid alkyl ester in the presence of a basic catalyst with formation of alcohol. If fatty acid amidoalkoxylates are replaced by fatty alcohol ethoxylates or glycols, for example propylene glycol, the interval between the addition of the catalyst to the reactants and the first formation of alcohol is considerably longer (Table 1).

TABLE 1
Time when the first methanol is separated off, as a function of the additive

| | | Methanol |
|---|---|---|
| Fatty acid N-methyl- | | separation |
| glucamide | Additive | in minutes |
| C₁₂-C₂₄-glucamide | Genagen CA 050 | 5 |
| C₁₆-C₁₈-glucamide | Genagen CA 050 | 6 |
| C₁₆-C₁₈-glucamide | Genagen PA 050 | 9 |
| C₁₆-C₁₈-glucamide | Genagen OA 100 | 13 |
| C₁₂-C₂₄-glucamide | monopropylene glycol | 18 |
| C₁₆-C₁₈-glucamide | Genapol LA 050 | 25 |
| C₁₆-C₁₈-glucamide | C₁₀-fatty acid dimethylamide | 31 |

Reaction temperature: 120° C., in the case of C₁₀-fatty acid dimethylamide being

used: 120-130° C.

Mixing ratio: amine/additive: 70/30

Catalyst: sodium methoxide solution in methanol

The product mixture obtained in this manner can be incorporated without further purification methods into formulations, for example in detergents and cleaners, cosmetic compositions, crop protection compositions, etc.

The resulting paste-like mixtures solidify at room temperature, giving solid to tacky-solid blocks which can be processed further, in a further process step, by adding additives customarily used in the preparation of detergents and cleaners and dishwashing detergents, cosmetic compositions and crop protection compositions, such as, for example, surfactants, solubilizers, antifoams, builders, carrier materials, salts and extenders, bleaches, optical brighteners, antiredeposition agents and bleach activators, adjuvants, drift control agents and biocides.

The ratio by weight of the fatty acid N-alkylpolyhydroxyalkylamides of the formula I and the fatty acid amidoalkoxylates of the formula IlIl can vary within wide limits and is generally from 90:10 to 10:90, preferably from 90:10 to 30:70, in particular from 80:20 to 50:50,% by weight.

The fatty acid N-alkylpolyhydroxyamides of the formula I or mixtures of fatty acid N-alkylpolyhydroxyamides of the formula I and fatty acid amidoalkoxylates of the formula IlIl prepared by the process according to the invention can generally be employed in detergents and cleaners of any type, preferably in manual dishwashing detergents, liquid all-purpose cleaners or liquid light-duty detergents for handwashing.

Furthermore, the products prepared according to the invention are suitable nonionic mild surfactants, and also emulsifiers for cosmetic cleansing and care compositions for skin and hair.

In addition, the products prepared according to the invention can be used in the crop protection and metal processing sectors.

### EXAMPLES

Hereinbelow, examples of the preparation of fatty acid N-alkylpolyhydroxyamides using fatty acid amidoalkoxylates are described in more detail, without the invention being limited to these examples.

Examples:

1. Preparation of C₁₆-C₁₈-alkyl-N-methylglucamide in the presence of coconut fatty acid amide ethoxylate and sodium methoxide 220.8 g (0.5 mol) of an aqueous solution of N-methylglucamine having an N-methylglucamine content of 94% by weight based on the solid substance were dewatered using a stripper. To this end, the solution was heated to 100° C. and initially dewatered at atmospheric pressure to a residual water content of about 5% by weight. The solution was then heated to 130° C. and kept at this temperature, and a reduced pressure of up to 30 mbar was applied stepwise. The compound was then dewatered further up to a residual water content of 0.2% by weight, based on the melt obtained. This melt, which contained 97.6 g (0.5 mol) of N-methylglucamine, was, at 130° C. and with stirring, admixed with 102.9 g of Genagen CA 050 (30% by weight, based on the total mixture), heated to 100° C., and 150.7 g (0.54 mol or 108 mol %, based on N-methylglucamine) of hexadecanoic/octadecanoic acid methyl ester (C₁₆ fraction 55-65%, C₁₈ fraction 30-45%), heated to 100C. 9.0 g or 10 mol %, based on N-methylglucamine, of sodium methoxide (30% strength by weight in methanol) were then added dropwise with stirring over a period of 5 minutes. The temperature of the reaction mixture decreased to 120° C. The first methanol was formed after only about 6 minutes. The elimination product was distilled off initially at atmospheric pressure and at from 115° C. to 100° C., and, after 10 to 15 minutes, at 60 mbar and at from 85° C. to 90° C. The pressure was reduced to 30 mbar and maintained until the formation of methanol had ended.

2. Preparation of C₁₂-C₁₄-alkyl-N-methylglucamide in the presence of coconut fatty acid amide ethoxylate and sodium methoxide

220.8 g (0.5 mol) of an aqueous solution of N-methylglucamine having an N-methylglucamine content of 94% by weight based on the solid substance were dewatered using a stripper. To this end, the solution was heated to 100° C. and initially dewatered at atmospheric pressure to a residual water content of about 5% by weight. The solution was then heated to 130° C. and kept at this temperature, and a reduced pressure of up to 30 mbar was applied stepwise. The compound was then dewatered further up to a residual water content of 0.2% by weight, based on the melt obtained. This melt, which contained 97.6 g (0.5 mol) of N-methylglucamine, was, at 130° C. and with stirring, admixed with 87.3 g of Genagen CA 050 (30% by weight, based on the total mixture), heated to 100° C., and 115.5 g (0.525 mol or 105 mol %, based on N-methylglucamine) of C₁₂-/C₁₄-fatty acid methyl ester (C₁₂ fraction 65-75%, C₁₄ fraction 20-30%, C₁₆ fraction 4-8%), heated to 100° C. 9.0 g or 10 mol %, based on N-methylglucamine, of sodium methoxide (30% strength by weight in methanol) were then added dropwise with stirring over a period of 5 minutes. The temperature of the reaction mixture decreased to 120° C. The first methanol was formed after only about 5 minutes. The elimination product was distilled off initially at atmospheric pressure and at from 115° C. to 100° C., and, after 10 to 15 minutes, at 60 mbar and at from 85° C. to 90° C. The pressure was reduced to 30 mbar and maintained until the formation of methanol had ended.

3. Preparation of C₁₆-C₁₈-alkyl-N-methylglucamide in the presence of palm oil fatty acid amide ethoxylate and sodium methoxide

220.8 g (0.5 mol) of an aqueous solution of N-methylglucamine having an N-methylglucamine content of 94% by weight based on the solid substance were dewatered using a stripper. To this end, the solution was heated to 100° C. and initially dewatered at atmospheric pressure to a residual water content of about 5% by weight. The solution was then heated to 130° C. and kept at this temperature, and a reduced pressure of up to 30 mbar was applied stepwise. The compound was then dewatered further up to a residual water content of 0.2% by weight, based on the melt obtained. This melt, which contained 97.6 g (0.5 mol) of N-methylglucamine, was, at 130° C. and with stirring, admixed with 102.9 g of palm oil fatty acid amide ethoxylate (30% by weight, based on the total mixture), heated to 100° C., and 150.7 g (0.54 mol or 108 mol %, based on N-methylglucamine) of hexadecanoic/octadecanoic acid methyl ester (C₁₆ fraction 55-65%, C₁₈ fraction 30-45%), heated to 100° C. 9.0 g or 10 mol %, based on N-methylglucamine, of sodium methoxide (30% strength by weight in methanol) were then added dropwise with stirring over a period of 5 minutes. The temperature of the reaction mixture decreased to 120° C. The first methanol was formed after only about 9 minutes. The elimination product was distilled off initially at atmospheric pressure and at from 1150C to 100° C., and, after 10 to 15 minutes, at 60 mbar and at from 85° C. to 90° C. The pressure was reduced to 30 mbar and maintained until the formation of methanol had ended.

4. Preparation of C₁₆-C₁₈-alkyl-N-methylglucamide in the presence of oleic acid amide ethoxylate and sodium methoxide

220.8 g (0.5 mol) of an aqueous solution of N-methylglucamine having an N-methylglucamine content of 94% by weight based on the solid substance were dewatered using a stripper. To this end, the solution was heated to 100° C. and initially dewatered at atmospheric pressure to a residual water content of about 5% by weight. The solution was then heated to 130° C. and kept at this temperature, and a reduced pressure of up to 30 mbar was applied stepwise. The compound was then dewatered further up to a residual water content of 0.2% by weight, based on the melt obtained. This melt, which contained 97.6 g (0.5 mol) of N-methylglucamine, was, at 130° C. and with stirring, admixed with 102.9 g of oleic acid amide ethoxylate (30% by weight, based on the total mixture), heated to 100° C., and 150.7 g (0.54 mol or 108 mol %, based on N-methylglucamine) of hexadecanoic/octadecanoic acid methyl ester (C₁₆ fraction 55-65%, C₁₈ fraction 30-45%), heated to 100° C. 9.0 g or 10 mol %, based on N-methylglucamine, of sodium methoxide (30% strength by weight in methanol) were then added dropwise with stirring over a period of 5 minutes. The temperature of the reaction mixture decreased to 120° C. The first methanol was formed after only about 13 minutes. The elimination product was distilled off initially at atmospheric pressure and at from 115° C. to 100° C., and, after 10 to 15 minutes, at 60 mbar and at from 85° C. to 90° C. The pressure was reduced to 30 mbar and maintained until the formation of methanol had ended.

5. Preparation of C₁₆-C₁₈-alkyl-N-methylglucamide in the presence of coconut fatty acid amide ethoxylate and NaOH

220.8 g (0.5 mol) of an aqueous solution of N-methylglucamine having an N-methylglucamine content of 94% by weight based on the solid substance, and 4.0 g of 50% strength by weight of aqueous sodium hydroxide solution (10 mol %, based on N-methylglucamine) were dewatered using a stripper. To this end, the solution was heated to 100° C. and initially dewatered at atmospheric pressure to a residual water content of about 5% by weight. The solution was then heated to 130° C. and kept at this temperature, and a reduced pressure of up to 30 mbar was applied stepwise. The compound was then dewatered further up to a residual water content of 0.2% by weight, based on the melt obtained. This melt, which contained 97.6 g (0.5 mol) of N-methylglucamine, was admixed dropwise over a period of about 15 minutes, at 130° C. and with stirring, with 102.9 g of Genagen CA 050 (30% by weight, based on the total mixture), heated to 100° C., and 150.7 g (0.54 mol or 108 mol %, based on N-methylglucamine) of hexadecanoic/octadecanoic acid methyl ester (C₁₆ fraction 55-65%, C₁₈ fraction 30-45%), heated to 100° C. The temperature of the reaction mixture decreased to 120° C. The first methanol was formed after only about 5 minutes. The elimination product was distilled off initially at atmospheric pressure and at from 115° C. to 100° C., and, after 10 to 15 minutes, at 60 mbar and at from 85° C. to 90° C. The pressure was reduced to 30 mbar and maintained until the formation of methanol had ended.

6. Preparation of C₁₆-C₁₈-alkyl-N-methylglucamide in the presence of lauryl alcohol ethoxylate and sodium methoxide

220.8 g (0.5 mol) of an aqueous solution of N-methylglucamine having an N-methylglucamine content of 94% by weight based on the solid substance were dewatered using a stripper. To this end, the solution was heated to 100° C. and initially dewatered at atmospheric pressure to a residual water content of about 5% by weight. The solution was then heated to 130° C. and kept at this temperature, and a reduced pressure of up to 30 mbar was applied stepwise. The compound was then dewatered further up to a residual water content of 0.2% by weight, based on the melt obtained. This melt, which contained 97.6 g (0.5 mol) of N-methylglucamine, was, at 130° C. and with stirring, admixed with 102.9 g of Genapol LA 050 (30% by weight, based on the total mixture), heated to 100° C., and 150.7 g (0.54 mol or 108 mol %, based on N-methylglucamine) of hexadecanoic/octadecanoic acid methyl ester (C₁₆ fraction 55-65%, C₁₈ fraction 30-45%), heated to 100° C. 9.0 g or 10 mol %, based on N-methylglucamine, of sodium methoxide (30% strength by weight in methanol) were then added dropwise with stirring over a period of 5 minutes. The temperature of the reaction mixture decreased to 120° C. The first methanol was formed after about 25 minutes. The elimination product was distilled off initially at atmospheric pressure and at from 115° C. to 100° C., and, after 10 to 15 minutes, at 60 mbar and at from 85° C. to 90° C. The pressure was reduced to 30 mbar and maintained until the formation of methanol had ended.

7. Preparation of C₁₂-C₁₄-alkyl-N-methylglucamide in the presence of oxalkylated coconut fatty acid monoethanolamide and sodium methoxide

220.8 g (0.5 mol) of an aqueous solution of N-methylglucamine having an N-methylglucamine content of 94% by weight based on the solid substance were dewatered using a stripper. To this end, the solution was heated to 100° C. and initially dewatered at atmospheric pressure to a residual water content of about 5% by weight. The solution was then heated to 130° C. and kept at this temperature, and a reduced pressure of up to 30 mbar was applied stepwise. The compound was then dewatered further up to a residual water content of 0.2% by weight, based on the melt obtained. This melt, which contained 97.6 g (0.5 mol) of N-methylglucamine, was, at 130° C. and with stirring, admixed with 87.3 g of Genagen CA 050 (30% by weight, based on the total mixture), heated to 100° C., and 115.5 g (0.525 mol or 105 mol %, based on N-methylglucamine) of C₁₂-/C₁₄-fatty acid methyl ester

(C₁₂ fraction 65-75%, C₁₄ fraction 20-30%, C₁₆ fraction 4-8%), heated to 100° C. 9.0 g or 10 mol %, based on N-methylglucamine, of sodium methoxide (30% strength by weight in methanol) were then added dropwise with stirring over a period of

5 minutes. The temperature of the reaction mixture decreased to 120° C. The first methanol was formed after about 18 minutes. The elimination product was distilled off initially at atmospheric pressure and at from 115° C. to 100° C., and, after 10 to

15 minutes, at 60 mbar and at from 85° C. to 90° C. The pressure was reduced to 30 mbar and maintained until the formation of methanol had ended.

Chemical names of the commercial products used:

| | |
|---|---|
| Genagen CA 050 | Coconut fatty acid monoethanolamide having 5 EO |
| Genagen PA 050 | Palm oil monoethanolamide having 5 EO |
| Genagen OA 100 | Oleic acid monoethanolamide having 10 EO |
| Genapol LA 050 | C₁₂-C₁₆ alcohol ethoxylate having 5 EO |

The products mentioned above are commercial products from Clariant GmbH, Frankfurt am Main.

1. A process for preparing fatty acid N-alkylpolyhydroxyamides of the formula
R¹CONR²Z
in which R¹ is a linear or branched, saturated or unsaturated alkyl group having 7 to 21 carbon atoms, Z is a polyhydroxyhydrocarbon group having at least three hydroxyl groups, which can also be alkoxylated, and R² is H, C₁-C₈-alkyl, a group of the formula —(CH₂)ₓNR³R⁴ or R⁵O(CH₂)ₙ—, R³ and R⁴ are C₁-C₄-alkyl or C₂-C₄-hydroxyalkyl, R⁵ is C₁-C₄-alkyl, n is a number from 2 to 4 and x is a number from 2 to 10, by reacting an N-alkyl-polyhydroxyamine of the formula
H—NR²Z
with a fatty acid, fatty acid chloride or fatty acid ester in the presence of a basic catalyst, which comprises carrying out the reaction in the presence of a fatty acid amidoalkoxylate of the formula
R—CONR⁶R⁷
where R is alkyl or alkenyl having 7 to 21 carbon atoms, R⁶ is hydrogen or a group —(AO)ₓH, R⁷ is a group —(AO)ₓH, A is a group of the formula —C₂H₄—, —C₃H₆— or —C₄H₈— and x is a number from 1 to 20.

2. The process as claimed in claim 1, wherein fatty acid N-alkylglucamides of the formula 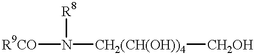
are prepared, where R⁸ is C₁-C₄-alkyl and R⁹CO is the radical of a saturated or unsaturated fatty acid having 12 to 18 carbon atoms.

3. The process as claimed in claim 1, wherein the reaction is carried out in the presence of a metal alkoxide, an alkali metal hydroxide or an alkali metal salt of glycerol or propylene glycol.

4. The use of the product mixture obtained according to claim 1 as surfactant or emulsifier in detergents and cleaners and in cosmetic preparations, in crop protection formulations and in preparations for processing metal.
